(19) United States
(12) Patent Application Publication
(10) Pub. No.: US 2021/0335490 A1
(43) Pub. Date: Oct. 28, 2021

(54) MAGNETIC RESONANCE IMAGING (MRI) SCAN WORKFLOW ASSISTANT

(71) Applicant: Siemens Healthcare GmbH, Erlangen (DE)

(72) Inventors: Silvia Bettina Arroyo Camejo, Fürth (DE); Tobias Kober, Lausanne (CH); Christoph Forman, Erlangen (DE)

(73) Assignee: Siemens Healthcare GmbH, Erlangen (DE)

(21) Appl. No.: 17/233,943

(22) Filed: Apr. 19, 2021

Related U.S. Application Data
(60) Provisional application No. 63/013,825, filed on Apr. 22, 2020.

(51) Int. Cl. G16H 50/20; G16H 30/40; A61B 5/055

(52) U.S. Cl. CPC G16H 50/20; A61B 5/055; G16H 30/40

(57) ABSTRACT

Techniques are disclosed for a scan recommendation method is disclosed, wherein evaluation data comprising classification data are received, the classification data classifying anomalies detected in input data concerning the medical condition of a patient. Based on the evaluation data, an automatic determination of a next recommended MR protocol to be executed in accordance with an MR scan of the patient is performed. Further, a personal scan preparation method is disclosed and a medical imaging method is described, as well as a scan workflow performing method. For execution of the above-mentioned methods, a scan recommendation system, a personal scan preparation system, a medical imaging system, and a scan workflow performing system are also described.

### CROSS-REFERENCE TO RELATED APPLICATIONS

This application claims priority to U.S. Provisional Application No. 63/013,825, filed on Apr. 22, 2020, the contents of which are hereby incorporated by reference their entirety.

### TECHNICAL FIELD

The present disclosure relates to techniques for developing a workflow for magnetic resonance imaging (MRI), and in particular to techniques for recommending MR protocols using various data sources, which may be part of an automated process.

### BACKGROUND

Magnetic resonance imaging (MRI) is a medical imaging technique that provides diagnostically relevant information of biological tissue. Its diagnostic relevance originates from the informative power and versatility of this imaging modality, which offers differentiated and precise structure information in a non-invasive fashion. Magnetic resonance imaging is based on the controlled manipulation of nuclear spins inside a patient's body and subsequent detection of the nuclear spin response. The space-dependent encoding of the spin response allows for a reconstruction of the patient's structural composition and functional constitution for the radiologist to perform diagnostic reading.

Conventionally, to generate MR images for diagnosis, the MRI technician conducting the MRI scan selects a sequential arrangement of MR protocol from an MRI scanner library of imaging protocols, which may be based, e.g., on a relevant body region, a suspected disease (e.g., through the clinical indication of the referring physician or the radiologist), patient-specific requirements (e.g., implants, motion-related incompliance), and preferences of the clinical institution and radiologists who will later perform reading of the MR images for diagnosis. In addition, during the scan procedure the technician typically performs checks to ensure that the image quality of the recorded scans allows for diagnosis. The technician may then make protocol modifications and will perform re-scans, if required.

This classical MRI scan workflow requires MRI technicians to undergo intense modality-specific training and acquire many years of scanning experience. Today, the number of performed MRI scans is continuously increasing while qualified personnel is scarce, which contributes to high operational costs. And even for highly trained technicians, the high number of manual steps required to optimally perform MRI scanning together with increasing time pressure causes significant risks for operational errors. Even in high profile hospitals, there might be times when less trained technicians have to operate the scanner, for example at night or on the weekend.

### SUMMARY

In U.S. Pat. No. 9,928,589 B2, an apparatus and a method for supporting acquisition of a multi-parametric image is described. The apparatus comprises a disease selector configured to select a suspected disease of a patient based on patient information; and an image selector configured to determine a set of imaging conditions of a multi-parametric magnetic resonance image corresponding to the suspected disease based on a multi-parametric magnetic resonance imaging model. However, it is often very difficult to determine the disease of a patient before having recorded medical images.

Furthermore, in U.S. Patent application Publication No. 2018/0101644 A1, a method for the provision of confidence information about a diagnosis of a diagnosis system based on medical image data is described. The diagnosis system is designed as an algorithm that uses as its input the medical image data and further patient specific data. The measure of confidence is determined by execution of a determination algorithm that has as its input parameter a characteristic value for the diagnosis system, for example a sensitivity or a specificity of the diagnosis system, and/or an image content from medical image data. Also a further acquisition of additional image data can be proposed based on the confidence information. The additional medical image data can be acquired such that the data exhibit a changed image contrast with respect to the already existing medical image data.

Thus, as of today, most MRI scans are performed manually, requiring the technician to select the required protocols from a protocol library. In an attempt to automate the MR imaging workflow, applications have been designed containing a sequential arrangement of protocols specifically composed for the imaging of a specific anatomical region and/or suspected disease. In particular, the steps of image slice planning can already be automated by means of pattern matching algorithms. However, as of today there is no software that can enable the untrained technician to perform an entire MRI scan with a high success rate while considering patient specific needs or findings.

Hence, it is an object of the embodiments of the present disclosure to find a solution for a preparation and execution of an MRI scan without the need of a qualified operating person. To do so, the above-mentioned problem is solved as described in accordance with the embodiments of the present disclosure and in the claims, examples of which include a scan recommendation method, a personal scan preparation method, a medical imaging method, a scan workflow performing method, a scan recommendation system, a personal scan preparation system, a medical imaging system, and a scan workflow performing system.

The scan recommendation method according to the embodiments of the disclosure comprises the step of receiving evaluation data comprising classification data. The classification data classify anomalies, which were detected based on input data concerning the medical condition of a patient. In an embodiment, the anomalies are detected in medical image data of the patient. A classification comprises a type of identification of a detected anomaly, assigning probability values for different possible types of anomalies to the detected anomaly. For example, the anomalies can be anomalies detected in the input data, for example medical image data, or the anomalies can comprise information about anomalies based on medical data about the patient of a different type.

Anomalies detected in image data comprise detected image abnormalities or distinctive image features such as hyperintense areas, hypointense areas, abnormal tissues, midline shift, vascular stenosis, plaques, demyelination, or mass effects. As explained further herein, anomalies may also be detected image metric abnormalities or distinctive features. Then, the next recommended MR protocol, which is intended to be executed with an MR scan of the patient, is automatically determined based on the evaluation data. As explained herein, the evaluation is not limited to the evaluation of image data. Hence, a plurality of different type of input data about a patient's condition can be analyzed to determine the evaluation data. Also, a plurality of data sets of a single special type of input data concerning a patient's condition can be evaluated. The scan recommendation can be based on receiving a plurality of evaluation data of different types. The detection of the anomalies can be realized based on machine learning methods, for example based on neural networks such as a convolutional neural network taking the input data, for example image data, as input and providing as output the probability of the input data to be normal versus anomalous. Also, the classification of the detected anomalies can be realized using machine learning methods, for example neural network-based techniques, such as a convolutional neural network for instance. If the probability value of a detected anomaly for the received input data, for example image data, is higher than a certain customizable threshold value a, then the input data (e.g. image data) assigned to the detected anomaly are taken as input for the subsequent classification step as mentioned above. The scan recommendation can also be realized based on machine learning based techniques, e.g. neural network techniques as explained herein. In one variant, the evaluation data simply include the classification data. However, the evaluation data may additionally comprise different types of data. The method according to an embodiment of the disclosure allows the trained or un-trained technician to perform a highly personalized MRI scan workflow on each patient, generating an optimal set of MRI images that are required for diagnosis of that patient's condition. The method may provide a basis for a fully autonomously optimized MR imaging workflow and yet allow for manual or electronic customization thereof as desired by the operator. The MR scanner operator may choose to run the MR scan workflow either in a fully autonomous mode, a fully manual mode, or in a hybrid mode that is a combination of both of these modes.

The personal scan preparation method according to an embodiment of the disclosure comprises the steps of receiving input data, for example medical image data, concerning the medical condition of a patient. Evaluation data based on the input data are determined by detecting anomalies in the input data, determining classification data based on the detected anomalies as evaluation data, and performing the scan recommendation method according to an embodiment of the disclosure. Advantageously, the classification of the anomalies can be used to adapt an MR protocol to a special kind of anomaly. For example, a special contrast is chosen for the recommended MR protocol. Further, the personal scan preparation method embodiment shares the advantages of the scan recommendation method embodiment.

The medical imaging method according to an embodiment of the disclosure comprises the steps of determining a next recommended protocol based on the scan recommendation method according to an embodiment of the disclosure, transmitting the next recommended protocol to an MR scan system, and performing an MR scan by the MR scan system based on the received protocol. The medical imaging method embodiment shares the advantages of the scan recommendation method embodiment.

The scan workflow performing method according to an embodiment of the disclosure comprises the steps of performing the medical imaging method an embodiment of the disclosure. After the MR scan of the medical imaging method, anomalies are detected in the reconstructed MR-image data and classification data are determined based on the detected anomalies. Then, the aforementioned three steps are iteratively carried out, taking into account the determined classification data, until an abort criterion is attained. In other words, first a next recommended protocol is determined based on the scan recommendation method embodiment of the disclosure or the personal scan preparation method embodiment of the disclosure. Then, the next recommended protocol is transmitted to an MR scan system and the MR scan is performed by the MR scan system based on the recommended protocol. Further, an iterative protocol determination and measurement is performed, comprising the steps of controlling the MR scan, evaluating the reconstructed MR-image data, and iteratively improving input data for the next recommended protocol based on classification of anomalies of the reconstructed MR-image data. For example, the operator can first record a standard set of MR protocol and employ the scan recommendation method based on the recorded first images for additional MR scans that might be additionally indicated for the current patient based on the imaging information.

Advantageously, a complete automatic workflow for an optimized MR scan of a patient is realized, wherein the protocol for the MR imaging and hence the MR imaging itself is optimized with each iteration loop. It is noted that all the methods embodiments described above, i.e. the scan recommendation method, the personal scan preparation method, the medical imaging method, and the scan workflow performing method, can also be carried out in the case that the operator does not sit in front of the scanner console. Hence, all of the described method embodiments can be performed without even a console room next to the MR scan system, i.e. fully automated, or in the event that the monitoring operator is sitting somewhere else and controls the MR scan system remotely. Advantageously, a skilled operator can perform an investigation of a patient and a surveillance of a medical imaging process remotely, and therefore professional skills can be accessed where an MR system is available.

The scan recommendation system according to an embodiment of the disclosure comprises at least one interface, e.g. a plurality of input interfaces, for receiving evaluation data. The evaluation data comprise classification data, wherein the classification data classify anomalies detected based on input data, wherein the input data concern the medical condition of a patient. Further, the scan recommendation system according to an embodiment of the disclosure includes a recommendation unit for automatic determination of a next recommended MR protocol to be executed in accordance with an MR scan for determining a next recommended MR protocol to be executed in accordance with an MR scan of the patient based on the received evaluation data.

In other words, the scan recommendation system can be configured to fully and automatically recommend the next, diagnostically most insightful MR protocol for measurement. The evaluation data for the recommendation system may be based on either a full set or subsets of inputs including image data (historical or current), image derived metrics, for example geometric properties of tissue and cavities like vascular wall thickness, cross section, and ventricular volume etc., electronic health record information, and/or user interface input. The scan recommendation system according to an embodiment of the disclosure shares the advantages of the scan recommendation method according to an embodiment of the disclosure.

The personal scan preparation system according to an embodiment of the disclosure comprises an input interface for receiving input data concerning the medical condition of a patient. Further, the personal scan preparation system includes an evaluation unit, which comprises a detection unit for detecting anomalies in the input data, a classification unit for determining classification data based on the detected anomalies as evaluation data, and the scan recommendation system according to an embodiment of the disclosure. The personal scan preparation system shares the advantages of the personal scan preparation method according to an embodiment of the disclosure.

The medical imaging system according to an embodiment of the disclosure comprises an MR scan system and a scan recommendation system or a personal scan preparation system according to an embodiment of the disclosure, which is coupled to the MR scan system to determine a next recommended protocol and to send the next recommended protocol to the MR scan system for performing an MR scan. The medical imaging system according to an embodiment of the disclosure shares the advantages of the medical imaging method according to an embodiment of the disclosure.

The scan workflow performing system comprises the medical imaging system according to an embodiment of the disclosure. Further, the scan workflow performing system includes an anomaly detection unit for detecting anomalies in the reconstructed MR-image data. Furthermore, the scan workflow performing system comprises a classification unit for determining classification data based on the detected anomalies. The scan workflow performing system also comprises an iteration unit for determining, based on the determined classification data, if iteratively performing the three steps of medical imaging, anomaly detection and anomaly classification, until an abort criterion is attained. The scan workflow performing system shares the advantages of the scan workflow performing method according to an embodiment of the disclosure.

The essential components of the scan recommendation system according to an embodiment of the disclosure, the personal scan preparation system according to an embodiment of the disclosure, the medical imaging system according to an embodiment of the disclosure, and the scan workflow performing system according to an embodiment of the disclosure can for the most part be designed in the form of software components. This applies for instance to the recommendation unit of the scan recommendation system, which can comprise for example a fully connected neural network or a random forest, the evaluation unit, the detection unit, and the classification unit of the personal scan preparation system and the anomaly detection unit and the iteration unit of the scan workflow performing system, but also parts of the input interfaces. In principle, however, some of these components can also be implemented in the form of software-supported hardware, for example FPGAs or the like, e.g. when particularly fast calculations are required or desirable. Likewise, the required interfaces, for example if it is only a matter of transferring data from other software components, can be designed as software interfaces. However, the required interfaces can also be designed as hardware-based interfaces that are controlled by a suitable software. Furthermore, some parts of the above-mentioned components may be distributed and stored in a local, regional, and/or global electronic network, or a combination of a network and software, e.g. a cloud system.

A largely software-based implementation has the advantage that medical imaging systems, including a magnetic resonance imaging system that have already been used, can easily be retrofitted by a software update to work in the manner according to embodiments of the disclosure. In this respect, the object is also achieved by a corresponding computer program product with a computer program that can be loaded directly into a memory device, for example a control device of a medical imaging system, with program sections, to carry out all steps of the method according to an embodiment of the disclosure, if the program is executed in the medical imaging system, e.g. the control device. In addition to the computer program, such a computer program product may contain additional components such as a documentation and/or additional components, including hardware components such as hardware keys (dongles etc.) for using the software.

[For transport to the medical imaging system and/or for storage on or in the medical imaging system, a computer-readable medium (e.g. a non-transitory computer-readable medium), for example a memory stick, a hard disk, or other suitable transportable or permanently installed data carrier may be used on which the program sections of the computer program that can be read in and executed by a computer unit of the medical imaging system are stored. The computer unit can comprise for example, one or more cooperating microprocessors or the like used for this purpose. The computer program can also be acquired by download from a central webshop of the Applicant or another Internet-based source or a cloud based source.

The disclosure as described herein contains particularly advantageous embodiments and developments. In particular, the claims of one claim category can also be further developed analogously to the dependent claims of another claim category. In addition, within the scope of the disclosure, the various features of different exemplary embodiments and claims can also be combined to form new exemplary embodiments.

In a variant of the scan recommendation method according to the disclosure, the automatic scan recommendation is based on using at least one of the following software types or types of a combination of software and hardware:
a machine learning model, e.g. including at least one of:
a shallow neural network model,
a deep neural network model,
a convolutional neural network,
a fully connected neural network,
a support vector machine,
a decision tree,
a random forest,
an expert system, and/or
a classical model algorithm.

For example, the scan recommendation method may be carried out using a trained model (for example a fully connected neural network or a random forest). The trained model receives the above-mentioned evaluation data, which may for example comprise classified anomalies Hence, the trained model is used to determine a next recommended MR protocol to be executed in accordance with an MR scan of a patient. The mentioned input data, for example image data of the patient, can be information of any suitable kind about the patient, which may influence an imaging process to be planned. The mentioned input data can either be directly used as input for the trained model or can undergo one or several preprocessing step(s), for example by a machine learning or deep learning model, before being fed into the trained model.

As an example, in the scan recommendation method according to the disclosure, the evaluation data comprise a first possible scan workflow direction determined by mapping topics, which are based on extracted keywords in the input data concerning a medical condition of the patient, onto probabilities of a next recommended protocol. The input data can be for example written information, i.e. text data about a suspected diagnosis, symptoms, an indication referring to the symptoms or a body region, where the symptoms are localized and to which the indication or suspected diagnosis refers. The input data can comprise electronic health record information comprising at least one of a suspected diagnosis, a suspected disease, relevant observations of the technician, an indication from referring clinician, a body region, patient data, or an MR protocol. Hence, electronic health record information can add additional information about the health condition of a patient and can be used for modifying an MR imaging protocol. Further, the protocol can be modified by direct input by the user. Therefore, the input data can also include data input by the user.

The keyword extraction can be performed based on an expert system or a machine learning approach or a deep learning approach, or combinations thereof. The extracted keywords can be determined based on data pre-processing, for example lemmatization, stemming, and/or bag-of words generation. The topics are determined by a topic detection algorithm or a generated topic model. The topic detection can be realized by a trained unsupervised topic detection with latent Dirichlet allocation. Latent Dirichlet allocation can be carried out using classical machine learning methods, for example a hierarchical Bayesian model trained with variational inference as described in D. Blei et al. JMLR 3, 2003, or using neural variational inference as described in Y. Miao et al. Proc. ICML, 2017. The mapping of the topics onto a possible scan workflow direction, i.e. a set of possible recommended next scan protocols, can be based on expert knowledge, customer preference, or an additional designated machine learning algorithm or deep learning algorithm. To each of the topics, a set of recommended next scan protocols with values of probabilities is assigned.

Further, a second possible scan workflow direction can be determined by the scan recommendation method according to the disclosure, wherein the classification data, which are included in the evaluation data, are mapped onto probability values of a next recommended protocol. Such a mapping can be realized using a mapping model, for example a random forest or a fully connected neural network. Then, the final recommendation step is performed based on the second possible scan workflow direction and, optionally, additionally based on the first possible scan workflow direction. The final recommending step can be realized using a weighted averaging of the first and the second possible scan workflow direction or using a fully connected neural network.

In a variant of the personal scan preparation method according to the disclosure, the input data comprise image data and the step of detecting anomalies comprises detecting anomalies in the image data. As mentioned above, the detection and classification of anomalies in image data can be realized using a neural network, for example a convolutional neural network. The anomalies can be direct anomalies of image data or anomalies of metrics related to image data. Therefore, the input data can also comprise image derived metrics based on image data and the step of detecting anomalies can comprise detecting anomalies in the image-derived metrics. Image derived metrics include geometric properties of tissues and cavities, potentially compared to normative data from a cohort. Such derived metrics encompass vascular wall thickness, cross sections of blood vessels and ventricular volume, grey mass volume in the brain, and white mass volume in the brain.

In the personal scan preparation method embodiment, the input data can also comprise electronic health record information and/or user interface input data, and the step of determining evaluation data can comprise the steps of extracting keywords from the electronic health record information and/or the user interface input, determining topics based on the extracted keywords and determining the first possible scan workflow direction by mapping the topics onto probabilities of a next recommended protocol. Keywords can comprise for example information regarding at least one of a suspected diagnosis, a suspected disease, relevant observations of the technician, an indication from referring clinician, a body region, patient data, or an MR protocol. The keyword extraction can be performed based on an expert system, a machine learning approach, a deep learning approach, or combinations thereof. The scan recommendation can be realized by one or several classical and or machine learning algorithms. As mentioned above, electronic health record information can add additional information about the health condition of a patient and can be used for modifying an MR imaging protocol. Further, the MR imaging protocol can be modified by direct input by the user. Using a keyword extraction enables an automatic analysis of electronic health record information and/or the user interface input concerning MR protocol related content. Advantageously, information concerning former medical examination can also be integrated into the scan recommendation based on written text or direct protocol data input by a user. In this way, the database for the automatic determination of a next recommended MR protocol is broadened.

As mentioned above, the different types of information can be combined as a base for automatic determination of a next recommended protocol. The aforementioned listed input data may include patient information, user information, and hospital information, which may either be directly used as input to the scan recommendation system or may undergo one or several preprocessing steps (e.g., by a machine learning or deep learning model) before being fed into the scan recommendation system.

In a variant of the scan recommendation system according to the disclosure, the recommendation unit comprises at least one of:
a machine learning model, including at least one of:
a shallow neural network model,
a deep neural network model,
a convolutional neural network,
a fully connected neural network,
a decision tree,
a random forest,
a support vector machine,
an expert system,
a classical model algorithm.

The described variant of a scan recommendation system embodiment of the disclosure shares the advantages of the corresponding variant of a scan recommendation method embodiment of the disclosure.

The scan recommendation system according to an embodiment of the disclosure can also comprise an input interface for receiving evaluation data comprising a first possible scan workflow direction determined by mapping topics based on extracted keywords concerning a medical condition of the patient onto probability values of a next recommended protocol. The described variant of a scan recommendation system embodiment of the disclosure shares the advantages of the corresponding variant of a scan recommendation method embodiment of the disclosure.

In an alternative variant of the scan recommendation system embodiment of the disclosure, the recommendation unit comprises a mapping unit for determining a second possible scan workflow direction by automated mapping of the classification data onto probability values of a next recommended protocol and a final protocol recommendation unit for final recommending the next recommended protocol based on the second possible scan workflow direction and optionally additionally based on the first possible scan workflow direction. The described variant of a scan recommendation system embodiment of the disclosure shares the advantages of the corresponding variant of a scan recommendation method embodiment of the disclosure.

In a variant of the personal scan preparation system according to the disclosure, the input interface is configured to receive input data comprising the medical image data and the detection unit is configured to detect anomalies in the image data. The input interface can also be configured to receive input data comprising image derived metrics based on image data, and the detection unit can be configured to detect anomalies in the image-derived metrics. The variants of the personal scan preparation system embodiment of the disclosure share the advantages of the corresponding variants of a personal scan preparation method embodiment of the disclosure.

In a further variant of the personal scan preparation system according to the disclosure, the input interface is configured to receive input data comprising electronic health record information and/or user interface input data. In accordance with such a variant, the evaluation unit comprises an extraction unit for extracting keywords from the electronic health record information and/or the user interface input, a topic finding unit for determining topics based on the extracted keywords, and a mapping unit for determining the first possible scan workflow direction by mapping the topics onto probability values of a next recommended protocol as evaluation data. The described variant of the personal scan preparation system embodiment of the disclosure shares the advantages of the corresponding variant of the personal scan preparation method embodiment of the disclosure.

### BRIEF DESCRIPTION OF THE DRAWINGS/FIGURES

The above and other features and advantages of the present disclosure will be more apparent to those of ordinary skill in the art from the detailed description of preferred embodiments of the present disclosure with reference to the accompanying drawings, in which:

FIG. 1 illustrates an example process flow of a scan preparation method according to an embodiment of the present disclosure;

FIG. 2 illustrates an example process flow providing further detail with respect to the process flow of the method illustrated in FIG. 1 according to an embodiment of the present disclosure;

FIG. 3 illustrates an example process flow of a scan workflow according to an embodiment of the present disclosure;

FIG. 4 illustrates an example block diagram showing a personal scan preparation system with a scan recommendation system according to an embodiment of the present disclosure;

FIG. 5 illustrates an example block diagram showing a medical imaging system with the personal scan preparation system as depicted in FIG. 4 according to an embodiment of the present disclosure;

FIG. 6 illustrates an example block diagram showing a scan workflow performing system according to an embodiment of the present disclosure;

FIG. 7 illustrates an example of an image data anomaly classification according to an embodiment of the present disclosure;

FIG. 8 illustrates an example of a metrics anomaly classification according to an embodiment of the present disclosure;

FIG. 9 illustrates an example of a keyword analysis based on electronic health record input data or user interface input data according to an embodiment of the present disclosure; and

FIG. 10 illustrates an example of the function of a scan recommendation according to an embodiment of the present disclosure.

### DETAILED DESCRIPTION

In order to make the object, technical solutions, and advantages of the present disclosure more apparent, the present disclosure will be further described in detail by way of embodiments hereinafter.

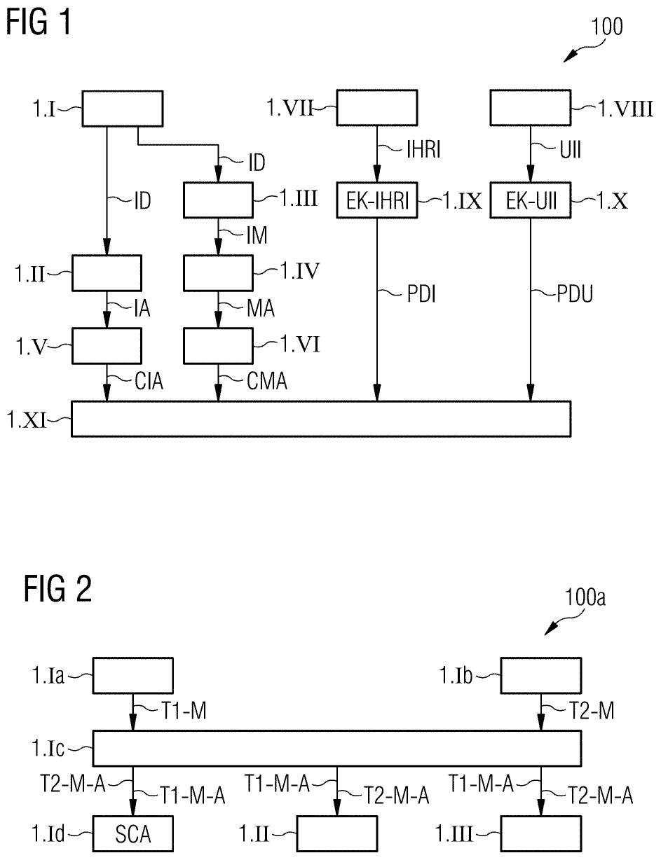

FIG. 1 illustrates an example process flow of a scan preparation method according to an embodiment of the present disclosure. The process flow 100 as shown in FIG. 1 corresponds to a scan preparation method for recommending a workflow for medical imaging of a patient. As shown in FIG. 1, in step 1.I of the scan preparation method, image data ID from the patient to be examined are acquired. The image data ID may be acquired by an initial scan for creating a T1-map and a T2-map. Further, the image data ID may be received from historical images previously acquired from the patient. In step 1.II, the image data ID are directly used as first input data for determination of evaluation data. The image data are analyzed regarding image anomalies IA. The detection of anomalies IA in the image data ID can be realized using a convolutional neural network, for instance, or any other suitable type of neural network of machine learning-based architecture. In step 1.III, the image data ID are first subjected to pre-diagnostic analysis steps, in which some metrics IM of the image data ID are derived. For example, some pre-diagnostic analysis data such as, for instance, geometric properties of tissue and cavities like vascular wall thickness, cross section, ventricular volume, etc. are detected and measured, and are compared to normative data from a cohort.

Further, in step 1.IV, these image metrics IM are used as second input data and are subjected to an anomaly detection using a fully connected neural network, for instance, in which some anomalies MA of metrics IM can be detected. In step 1.V, the image anomalies IA are further classified using, for example, the above-mentioned convolutional neural network. In step 1.VI, the detected anomalies MA of metrics IM are classified using, for example, a fully connected neural network, a decision tree, a random forest, etc. Hence, in step 1.V and step 1.VI, evaluation data comprising classification data CIA, CMA for image anomalies IA and metrics anomalies MA are provided for later step 1.XI, which is the actual scan recommendation step. The classification data CIA concerning image anomalies IA comprise sets of probability values P(i) for the probability of the existence of different image anomalies IA in the image data ID input to the scan preparation method in step 1.I. The classification data CMA concerning metrics anomalies MA comprise sets of probability values P(j) for the probability of the existence of different metrics anomalies IM, which can also be understood as input data provided to the scan preparation method in step 1.IV.

In other words, the initial image data ID are recorded to provide a first measurement step benchmark and subsequently input these image data ID for an anomaly detection and anomaly classification. Quality assurance steps following each measurement can be optionally implemented to ensure sufficient image quality and trigger rescans, if required (e.g., due to patient motion). The anomaly detection and anomaly classification may be implemented, e.g., by a convolutional neural net, optionally as independent implementations. If anomaly detection/classification is applied, embodiments include advantageously using quantitative MR imaging protocols, for example T1-maps and T2-maps for the measurement of the initial input images to provide a standardized image input for the anomaly detection and classification.

In step 1.VII, any suitable type of electronic health record information IHRI is used as third input data, which may comprise data from various sources such as objects, for example suspected diagnoses, referring indications, etc., and/or other data sources such as a radiological information system (RIS), patient data, etc. In step 1.VIII, some additional information UII, also referred to as user interface input UII that comprises data indicating, for example, suspected diagnoses, referring indications, a body region(s), a desired MR protocol, etc., are acquired as fourth input data for the personal scan preparation method. In step 1.IX, the input electronic health record information IHRI is processed using, for example, natural language processing (NLP) to perform keyword extraction. In this way, extracted keywords EK-IHRI of input electronic health record information IHRI are acquired.

Further continuing the current example of the personal scan preparation method, the user interface input UII is processed using, for example, NLP to additionally or alternatively extract keywords EK-UII in step 1.X. As above-mentioned and later described in more detail herein, the extracted keywords EK-IHRI, EK-UII are then assigned to different topics, and the topics are then mapped to sets of probability values P(n), P(o), which represent a possible scan workflow direction PDI, PDU. Then, the possible scan workflow directions PDI, PDU are provided as third and fourth evaluation data, respectively, to the automatic scan recommendation method of step 1.XI. In step 1.XI, an automatic scan recommendation according to an embodiment of the disclosure is performed based on a combination of various evaluation values CIA, CMA, PDI, and PDU to identify the next recommended protocol, as further discussed herein. The automatic scan recommendation can be realized based on a classical or machine learning system, such as a fully connected neural network (FCNN), for instance. However, the scan recommendation is not limited to this implementation and may be implemented in accordance with any suitable type of classical algorithm, expert system, or machine learning design that may process inputs, e.g. in accordance with classical, training, or learning processes to recommend protocols based upon specific type of inputs.

FIG. 2 illustrates an example process flow providing further detail with respect to the process flow of the method illustrated in FIG. 1 according to an embodiment of the present disclosure. As shown in FIG. 2, the process flow 100a, illustrates step 1.I as shown in FIG. 1 in further detail. In step 1.Ia, a T1-map measurement is carried out based on an initial MR scan of the patient, and a T1-map T1-M is acquired. The initial scan of the patient can be done with low resolution, for instance, and thus does require much time compared to the main imaging process of the examination. Further, in step 1.Ib, a T2-map measurement is carried out based on the initial MR scan of the patient, and a T2-map T2-M is acquired. In step 1.Ic, a quality analysis of the acquired data T1-M, T2-M is performed and some quality proved image data T1-M-A, T2-M-A are provided for further processing and analysis. In Step 1.Id, the acquired data T1-M-A, T2-M-A, which are comprised by the image data ID mentioned in context with step 1.I, are used for generation of synthetic contrast images SCA. Further, the acquired data T1-M-A, T2-M-A are also used as image data ID in steps 1.II and 1.III as discussed above with respect to FIG. 1.

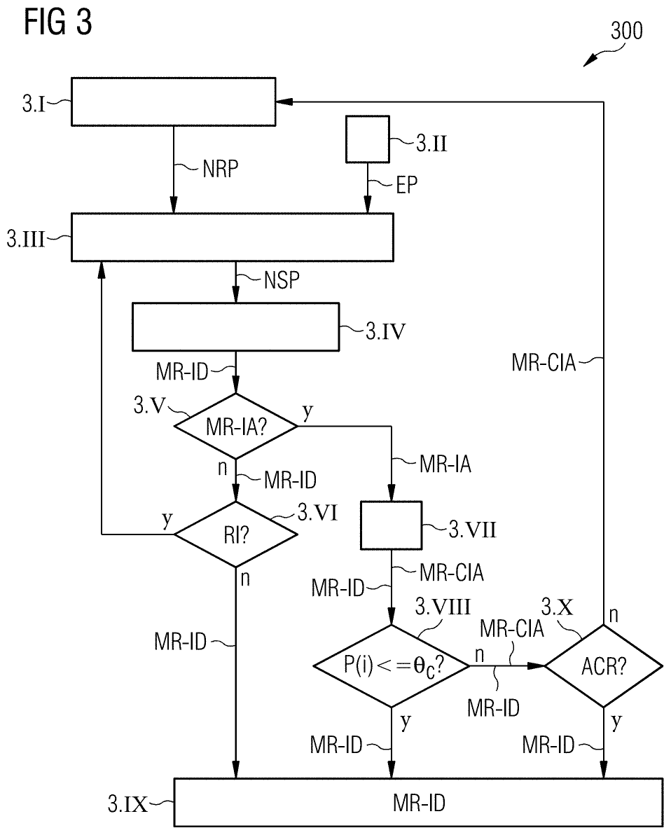

FIG. 3 illustrates an example process flow of a scan workflow according to an embodiment of the present disclosure. As shown in FIG. 3, the process flow 300 is depicted, which illustrates a scan workflow performing method comprising the above-mentioned personal scan preparation method including a scan recommendation of a workflow for a scan, and an additional iterative protocol determination and measurement process that may run until a relevant scan abort criterion combination is fulfilled. The process flow 300 begins with step 3.I, which includes a complete personal scan preparation method as explained in detail above with respect to FIG. 1, comprising step 1.I to step 1.XI. Hence, in step 3.I the method as described in FIG. 1 is performed and a next recommended protocol NRP is created based on the input data ID, IM, IHRI, and UII, which are analyzed and processed as described above with respect to FIG. 1. The next recommended protocol NRP is put into (e.g. stored in) a scan queue.

Further, in step 3.II, some additional manually- and/or electronically-selected protocols EP to be scanned are added to the scan queue due to, for example, clinical standards, health insurance guidelines, radiologist or technician preferences, etc. Then, in step 3.III, the next scan protocol NSP is transmitted from the scan queue to an MR scan unit. In step 3.IV, the actual scan process for the examination of the patient and acquisition of raw data as a basis for the reconstruction of image data MR-ID with high resolution and quality from a patient is performed. In step 3.V, the reconstructed image data MR-ID are then analyzed concerning potential anomalies MR-IA in the image data MR-ID. In the event that some anomalies MR-IA are detected (‘y’), the process continues with step 3.VII. In the event that no anomalies MR-IA are detected (‘n’), then the process continues with step 3.VI. In step 3.VI, it is determined if any additional item RI remains in the queue, which means that a protocol remains in the queue, which still needs to be executed. If this is the case (‘y’), the process continues with step 3.III. In case no additional item RI remains in the queue (‘n’), then the process continues with step 3.IX, i.e. the scan is completed and the reconstructed image data MR-ID are transmitted to the medical personal.

If the process continues with step 3.VII, a classification of the detected anomalies MR-IA is performed, for example using a fully connected neural network. In step 3.VIII it is determined if the predicted probabilities P(i) of the classes i of the classified anomaly data MR-CIA are each limited by a tunable probability threshold value θc. If this is the case (‘y’), the process continues with step 3.IX. In the event that at least one of the probabilities P(i) of the classes i exceeds the tunable probability threshold value θc (‘n’), then the process continues with step 3.X, in which it is checked if any scan abort flags were raised requiring the completion of the scan workflow. An abort flag can be raised based on, for instance, an abort criterion ACR, which can be a time limit that is reached, a maximum number of scan protocols reached, no scan protocol remaining in the scan queue, etc. If no abort criterion ACR is satisfied (‘n’), then the process continues with step 3.I, in which the detected anomalies MR-CIA are taken into account for the scan recommendation of the next MR scan. In the event that at least one of the abort criteria ACR is satisfied (‘y’), then the process ends with step 3.IX.

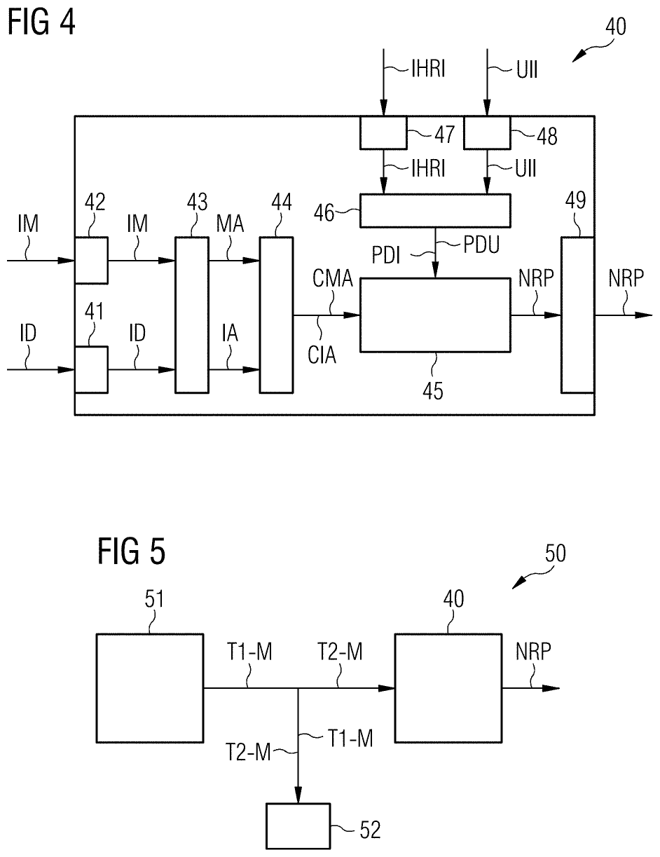

FIG. 4 illustrates an example block diagram showing a personal scan preparation system with a scan recommendation system according to an embodiment of the present disclosure. FIG. 4 shows a personal scan preparation system 40, having various blocks or modules as shown and described herein, which may be implemented as any suitable number of hardware circuitry, processors (e.g. processing circuitry), and/or software (e.g. executed algorithms) The personal scan preparation system 40 comprises an image data input interface 41, which receives the above-mentioned image data ID, for example, from an initial scan. Further, the scan recommendation system 40 comprises a metrics data input interface 42, which receives image metrics IM. Furthermore, the personal scan preparation system 40 comprises an anomaly detection unit 43, which is configured to detect some anomalies IA in the image data ID and some anomalies MA in the image metrics IM. The image anomalies IA and metrics anomalies MA are transmitted to an anomaly classification unit 44, which is configured to classify the detected anomalies IA, MA and which transmits the detected classification CIA, CMA to a scan recommendation unit 45.

In one variant, the anomaly detection unit 43 may perform the anomaly detection using a convolutional neural network configured to process the initial images ID as an input, and to provide as output the probability of the input images ID to be normal (i.e., healthy) vs. anomalous (i.e. requiring further imaging). If the probability of anomaly IA for the provided input image ID is higher than a certain, customizable threshold value fid, the initial input image ID with marked anomalies IA may then be inserted into (e.g. transmitted or otherwise output to) the subsequent anomaly classification unit 44, which classifies the anomalies IA into categories i with probability values P(i) for different classes that serve a meaningful input to the scan recommendation unit 45. Suitable classification categories i may include, for instance, one or several of the following exemplary class groups: disease classes (tumor, hemorrhage, MS, etc.), anomalous anatomical regions (white matter, dark matter, brain stem, CSF, etc.), anomalous metrics (white matter volume, vascular wall thickness, carotid cross section, etc.). The classes i with high predicted classification probability P(i) are then forwarded as one of the inputs to the scan recommendation unit 45. A selection of which classes are being forwarded to the scan recommendation unit 45 may be performed, e.g. by selecting a custom threshold probability value ξc, such that all classes i are forwarded the probability P(i) of which satisfy P(i)>ξc. In another variant, the forwarding criterion for a class i may alternatively be set to satisfy

$${max}_{i} P ( i ) \qquad$$

### and P(i)>ξc.

Additionally, the personal scan preparation system 40 also comprises a third input interface 47 for receiving electronic health record information IHRI. These electronic health record information IHRI of a patient can include for example known diseases, symptoms, clinical indications for the scan, contraindications (e.g. contrast intolerances), etc., and can be retrieved for example from the RIS or another electronic system containing patient data. Furthermore, the personal scan preparation system 40 includes a manual input interface 48 as a fourth input interface for receiving any suitable user interface input UII, for example containing suspected disease, or manually selected protocols to be included in the scan workflow. The additionally received data IHRI, UII are transmitted to a keyword extraction unit 46, which extracts some relevant keywords EK-IHRI, EK-UII from the received data IHRI, UII. Further, the keywords are assigned to topics, which are mapped to possible scan workflow directions PDI based on the electronic health record information IHRI and possible scan workflow directions PDU based on the user interface input UII as evaluation values. These evaluation values are then also transmitted to the scan recommendation system 45.

The scan recommendation system 45 performs an automatic scan recommendation based on a combination of various inputs CIA, CMA, PDI, PDU to identify a next recommended protocol NRP. The identified next recommended protocol NRP is output by an output interface 49, which is also part of the personal scan preparation system 40. In various embodiments, the personal scan preparation system 40 may be implemented as any suitable number of hardware circuitry, processors (e.g. processing circuitry), and/or software (e.g. executed algorithms) As an example, the scan recommendation system 40 may be implemented as any suitable type of classical or machine learning system, such as a fully connected neural network (FCNN). In an embodiment, the personal scan preparation system 40 may work in conjunction with other classical or machine learning processes, processors, hardware components, and/or interfaces, and/or additional executed algorithms to receive data via any suitable number of inputs. For instance, the personal scan preparation system 40 comprises four input interfaces 41, 42, 43, 44 and utilizes four inputs ID, IM, IHRI, UII. Although this is by way of example and not limitation. Embodiments include the personal scan preparation system 40 being adapted to any suitable number of inputs that may be greater than or less than four inputs and may include alternate inputs than the examples shown in FIG. 4. In various embodiments, each of the input interfaces of the scan recommendation system 45 may be implemented using any suitable number of hardware circuitry, processors (e.g. processing circuitry), and/or software (e.g. executed algorithms) In various embodiments, each of the input interfaces of the scan recommendation system 45 may be implemented as a system (e.g. a classical or machine learning system) that is best or preferably configured in accordance with the particular type of data that is received at each respective input interface.

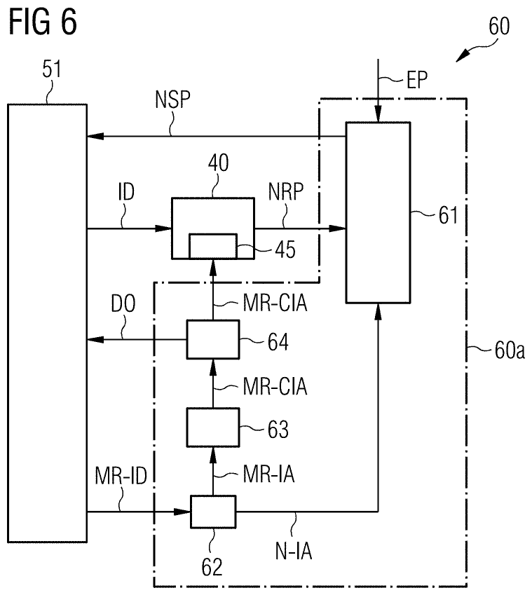

The scan recommendation system 45 may be implemented based upon, e.g., an expert system, a machine learning or deep learning approach, or combinations thereof. The scan recommendation algorithm of the scan recommendation system 45 may be realized, for example, by one or several classical or machine learning algorithms that are able to accept the evaluation data (which were predicted by the anomaly classification units and keyword extraction units upstream) and then predict the most appropriate scan protocol to be added to a scan queue (as shown in FIG. 6). As an example, an approach based on natural language processing with suitable word embeddings could be appropriate for this task. In an embodiment, the scan recommendation system 45 may be designed/trained, e.g., based on clinical expert knowledge and/or medical society guidelines.

FIG. 5 illustrates an example block diagram showing a medical imaging system with the personal scan preparation system as depicted in FIG. 4 according to an embodiment of the present disclosure. As shown in FIG. 5, a medical imaging system 50 is schematically illustrated, comprising the personal scan preparation system 40, explained in detail above with respect to FIG. 4. The medical imaging system 50 also comprises an MR scan system 51, which provides the personal scan preparation system 40 with T1-map data T1-M and T2-map data T2-M. Further, the medical imaging system 50 includes a contrast image generation unit 52, which generates based on the T1-map data T1-M and T2-map data T2-M, and some synthetic contrast images for the use by a radiologist who is used to a certain type of image with a certain contrast. The scan recommendation system of the personal scan preparation system 40 proposes a next recommend protocol NRP for an MR imaging process.

FIG. 6 illustrates an example block diagram showing a scan workflow performing system according to an embodiment of the present disclosure. As shown in FIG. 6, a block diagram is depicted showing a scan workflow performing system 60 with a personal scan preparation system 40 according to an embodiment, and an additional iterative protocol determination and measurement system 60a that may run until a relevant scan abort criterion ACR combination is met. The iterative protocol determination and measurement system 60a comprises a scan queue managing unit 61, which receives a next recommended protocol NRP from the scan recommendation system 45 of the personal scan preparation system 40 and which further receives some manually and/or electronically selected protocols EP to be scanned, and generates a scan queue, which comprises the received protocols NRP, EP.

In addition to the data-driven new recommended protocol NRP, embodiments include adding protocols to the scan queue manually and/or electronically. This option might be required to make a scan workflow compliant, e.g., with local hospital standards, health insurance provider requirements, medical society guidelines, user preferences, etc. Hence, scan protocols NRP, EP from both channels, i.e. the personal scan preparation system 40 and the manual/electronic input, are fed into a scan queue managing unit 61. The scan queue managing unit 61 then orders and/or curates the scan protocol jobs NRP, EP according to, e.g., scheduling time, relevance, redundancies, or the input channel. The scan queue managing unit 61 may (dependent on operator and user preferences) accept both protocol input channels or, in two extreme cases, reject one or the other input channels to enforce a manual or an automatic mode on a high-level. Dependent on operator and user preferences, the scan queue managing unit 61 may be configured to add only certain protocols, protocols with certain parameters, or protocols satisfying specific requirements, to the scan queue. The protocols in the scan queue may be executed during the next MR scanner measurement, resulting in a reconstructed MR image. A quality assurance step and an optional rescan (if indicated) may be inserted at this point or before image reconstruction, when applicable.

Therefore, the iterative protocol determination and measurement system 60a further comprises an anomaly detection unit 62, which is configured to detect anomalies MR-IA in the MR-image data MR-ID received from an MR scan system 51. The anomaly data MR-IA are transmitted to an anomaly classification unit 63, which is also part of the iterative protocol determination and measurement system 60a. Hence, the readily reconstructed image MR-ID is inserted into the anomaly detection unit 62 (e.g., similar in function as the anomaly detection unit 43 described above) suited for the respective MR scan protocol. If an anomaly MR-IA is detected, then the anomaly MR-IA may be classified by the above-mentioned subsequent anomaly classification unit 63, e.g. similar in function as the anomaly classification unit 44 described above.

The anomaly classification unit 63 arranges the anomaly data MR-IA in anomaly classes MR-CIA. The also referred to classification data MR-CIA are transmitted to an iteration unit 64, which determines based on the classification data MR-CIA and on a predetermined criterion ACR, whether the scan is completed or an additional MR scan has to be planned based on the classified anomalies MR-CIA. In case the probability P(i) of the anomaly classes i of the classification data MR-CIA is smaller than a threshold value θc, the scan is completed. If the probability P(i) of at least one of the anomaly classes i is higher than the threshold θc, it is determined whether an abort criterion ACR is satisfied, which can be a time limit that is reached, a maximum number of scan protocols reached, no scan protocol being left in queue, etc. In case one of these abort criteria ACR is satisfied, the scan is completed. In case the abort criteria ACR are all not satisfied, a new recommended protocol NRP is created based on the detected anomalies MR-CA. In this way, the classification data MR-CIA can be used as evaluation data for the scan recommendation system 45 of the scan preparation system 40 and a further MR scan is carried out using a newly recommended scan protocol.

As an example, following the anomaly classification, a step might be recommended that checks if any scan abort flags were raised requiring a completion of the scan workflow, e.g. due to a time limit being reached, a maximum number of scan protocols being reached, no scan protocols being left in the queue, etc. If no scan workflow abort criterion ACR is positive (i.e. identified), then the scan recommendation system 45 can be queried again with the classification data MR-CIA of the new scan image data MR-ID for one or several additional scan recommendation loops until one or a relevant combination of the following criteria ACR are met, which leads to a scan workflow completion:

1. No additional anomaly MR-IA is detected.

2. The classification probabilities P(i) of all anomaly classes i fall below a certain threshold

P(i)≤θc.

3. No item is left in the scan queue.

4. Any or a relevant combination of the scan workflow abort criterion ACR is positive.

In case the iteration unit 64 decides that the scan has to be completed, then an order DO is transmitted to the MR scan unit 51 for transmitting the image data MR-ID to the medical personnel.

In case the anomaly detection unit 62 does not detect an anomaly MR-IA in the MR image data MR-ID from the MR scan system 51, an information N-IA is sent to the scan queue managing unit 61, which determines if there is an additional item remaining in the queue. Then the scan queue status may decide if another pending scan will be executed next or if the scan workflow is completed. In the first case, the scan queue managing unit 61 continues with the next protocol in the queue.

Although the repeatedly-appearing configuration of an anomaly detection unit with an anomaly classification unit connected in series is described herein, this is not the only practical configuration and is provided by way of example and not limitation. The embodiments described herein may alternatively feed an image, image metrics, or other suitable data into the anomaly detection unit and the anomaly classification unit simultaneously for later combination of the results of both modules.

Moreover, the embodiments also include the modification of the thresholds for anomaly detection and anomaly classification (e.g., ξd, ξc, ϑd, ϑc) to different optimal values depending, e.g. on the position of the respective unit inside the scan workflow performing system 60, the scan protocol NSP employed for image generation, as well as other factors, a particular application, etc.

The methods and systems described herein allow for a trained or an untrained technician to perform a highly-personalized MRI scan workflow on each patient and to generate an optimal set of MRI images that are required for diagnosis of that patient's condition(s) considering potential anomalies detected on the recorded image data, electronic health record information, as well as manually- or electronically-set requirements from the side of the operator. These techniques as discussed herein may thus provide a basis for a fully autonomous and optimized MR imaging workflow that still allows for manual or electronic customization thereof as desired by the operator. The MR scanner operator may choose to run the MR scan workflow either in fully autonomous, fully manual, or in a hybrid mode that represents any suitable combination of both modes.

In an autonomous mode, dependent on the operator's preferences, by virtue of setting appropriate threshold values ξc, ϑc, and selecting relevant scan interruption criteria ACR, the MR scan workflow may be configured to, e.g., minimize the overall scan time per patient and/or to minimize the probability of missing a relevant MR protocol required for image reading and diagnosis.

In another variant, the operator may first record a standard set of MR protocols (e.g. by means of setting workflow step 3.II, 3.III in FIG. 3 appropriately) and employ the scan recommendation system 45 on demand to determine and record any additional MR scans that might be additionally indicated for the current patient.

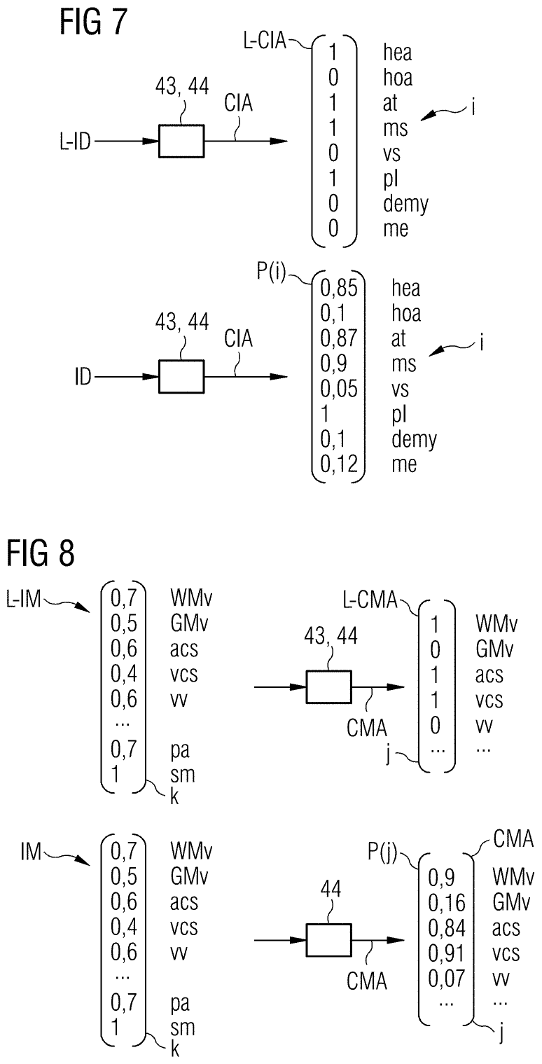

FIG. 7 illustrates an example of an image data anomaly classification according to an embodiment of the present disclosure. As shown in FIG. 7, an example for an image data anomaly classification is illustrated for a more detailed understanding. In the upper part of FIG. 7, a training process of an image data anomaly classification is depicted. Firstly, labelled image data L-ID are input as training data to a combined anomaly detection and classification unit 43, 44. The combined anomaly detection and classification unit 43, 44 comprises a convolutional neural network in this example, which creates classification data CIA as an answer to the labelled image data L-ID. Then, the classification data CIA are compared with the labelled classification data L-CIA and the convolutional neural network is adapted such that the classification data CIA are assimilated to the labelled classification data L-CIA.

Also shown in FIG. 7, some classes of image abnormalities IA are depicted, for example hyperintense areas hea, hypointense areas hoa, abnormal tissues at, midline shift ms, vascular stenosis vs, plaques pl, demyelination demy, and mass effects me. The anomaly classification data can be represented, for instance, as a 1×i-vector. Alternatively, tissue specific abnormalities or anomalies IA can also be used for anomaly classification. In that case, the anomalies IA are categorized by the tissue type and the anomaly type. For example, a tissue type for the brain can be white matter, grey matter, Hippocampus, Cerebellum or Corpus Callosum. The anomaly type can be hypertrophic, atrophic, or abnormal morphology. In the lower part of FIG. 7, a classification process is described. Firstly, image data ID are input to the anomaly detection and classification unit 43, 44. The trained convolutional neural network of the anomaly detection and classification unit 43, 44 now outputs classification data CIA comprising a 1×i-vector including probability values P(i) for the above-mentioned abnormalities IA.

FIG. 8 illustrates an example of a metrics anomaly classification according to an embodiment of the present disclosure. As shown in FIG. 8, an example for a metrics anomaly classification is illustrated for a more detailed understanding. In the upper part of FIG. 8, a training process of a metrics anomaly classification is depicted. Firstly, labelled metrics data L-IM are input to an anomaly detection and classification unit 43, 44 as training data. The anomaly detection and classification unit 43, 44 comprises a convolutional neural network in this example, which creates classification data CMA as an answer to the labelled metrics data L-IM. Then, the classification data CMA are compared with the labelled classification data L-CMA and the convolutional neural network is adapted such that the classification data CMA are assimilated to the labelled classification data L-CMA.

Also shown in FIG. 8, the metrics data IM comprise values concerning WMv volume, GMv volume, arterial cross section acs, venous cross section vcs, ventricular volume vv, and additional information about patient age pa and sex male sm of a patient. The metrics data IM can be represented as a 1×k-vector, and the anomaly classification data CMA can be represented by a 1×j-vector, in which case j=k−2 since the classification data CMA does not include information about age and sex of the patient. In the lower part of FIG. 8, a classification process is described. Firstly, metrics data IM are input to the anomaly detection and classification unit 43, 44. The trained convolutional neural network of the anomaly detection and classification unit 43, 44 now outputs classification data CMA comprising a 1×j-vector including probability values P(j) for the above-mentioned abnormalities MA.

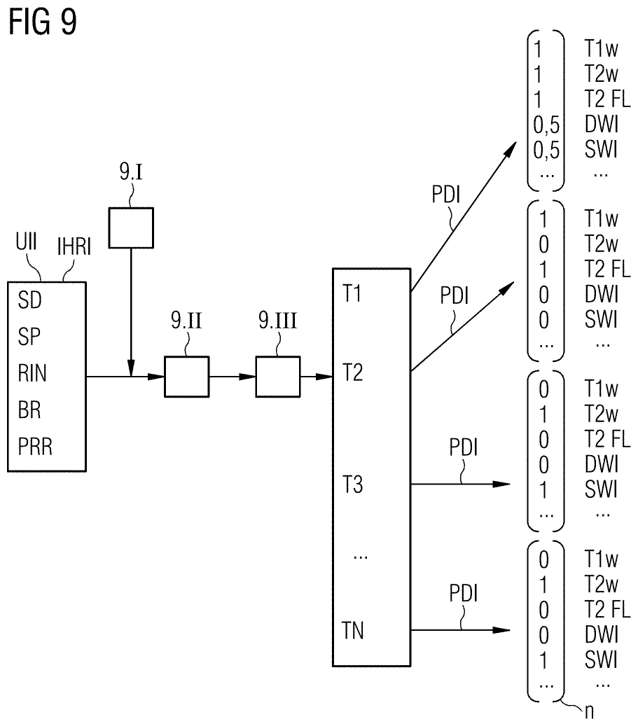

FIG. 9 illustrates an example of a keyword analysis based on electronic health record input data or user interface input data according to an embodiment of the present disclosure. As shown in FIG. 9, an example for a keyword analysis based on electronic health record input data IHRI or user interface input data UII is illustrated in detail. The electronic health record input data IHRI can comprise information about suspected diagnosis SD, symptoms SP, referring indication RIN, the assigned body region BR, and the MR protocol requested PRR. The keyword extraction starts with a data preprocessing step 9.I, for example a lemmatization and stemming. Then, in a step 9.II a bag-of-words generation is carried out. Stemming is a NLP (NLP=natural language programming) data preprocessing step, by which morphological variants (declensions and conjugations) of a word are traced back to the root word (stumbled→stumble). Lemmatization is an important NLP data preparation step, which reduces words from different inflectional forms to their base form (better→good). Bag-of-words generation aims at generating an unsorted catalogue of preprocessed, cleaned words (see above) from a text document, in which the frequency of the words is resolved.

After that, in step 9.III a topic detection based on a topic model is performed, wherein different topics T1, T2, T3, . . . , TN are detected. Then each identified topic, which is a set of keywords, is mapped to a possible scan workflow direction PDI, which is a set of recommended next scan protocols T1w, T2w, T2 FL, DWI, SWI. This mapping can be based for instance on expert knowledge, customer preference, or an additional, designated machine learning or deep learning based algorithm. In FIG. 9, the set PDI of recommended next scan protocols is represented by a 1×n-vector. The set PDI of recommended next scan protocols comprises, for example a T1 weighted protocol T1w, a T2 weighted protocol T2w, a T2 FLAIR protocol T2 FL, a diffusion weighted imaging protocol DWI, and a susceptibility weighted imaging protocol SWI. As another possibility, if no human mapping from topics to protocols is desired or possible, topic detection 9.III may be skipped and instead a model trained, e.g., a convolutional neural network, that predicts protocols directly from step 9.II. This is more direct, but offers less explanation to the user.

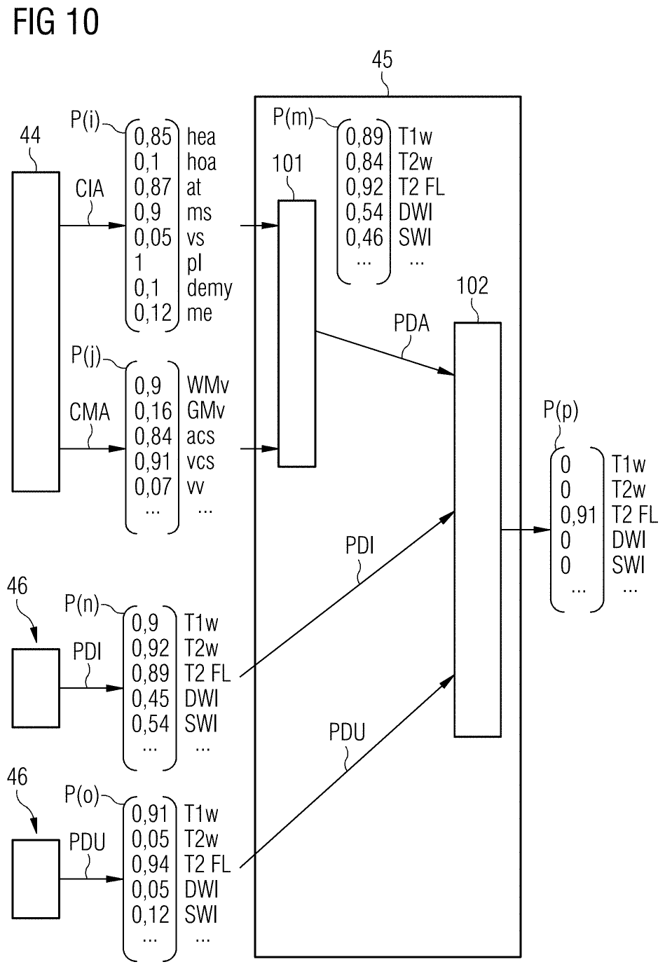

FIG. 10 illustrates an example of the function of a scan recommendation according to an embodiment of the present disclosure. As shown in FIG. 10, the function of a scan recommendation 45 is illustrated in detail. On the left side an anomaly classification unit 44 for classification of image anomalies IA and metrics anomalies MA is depicted. Further, keyword extraction units 46 for extracting keywords from electronic health record information data IHRI and user input data UII are depicted. As explained above with reference to FIGS. 7, 8, and 9, different classification data CIA, CMA are output by the classification unit 44 and possible scan workflow direction data PDI, PDU are output by the keyword extraction units 46. These data are input as evaluation data into the scan recommendation system 45.

The anomaly classification data CIA, CMA are then processed by a mapping unit 101, which functions based on a protocol mapping model, for example a random forest model or a fully connected neural network. In this manner, some probability values P(m) of next recommended protocols or possible scan workflow directions PDA are created, which have a similar format as the scan workflow direction data PDI, PDU. All these recommended next protocol data PDA, PDI, PDU are then input into a final protocol recommendation unit 102, which determines a final recommended protocol NRP by using for instance weighted averaging, a random forest model, a fully connected neural network, etc. As a result, a high probability value P(p=3) for a T2-FLAIR protocol T2 FL is determined. Hence a T2-FLAIR protocol T2 FL is recommended for the next MR scan.

The above descriptions are merely preferred embodiments of the present disclosure but not intended to limit the present disclosure, and any modifications, equivalent replacements, improvements, etc. made within the spirit and principle of the present disclosure should be included within the scope of protection of the present disclosure.

What is claimed is:

1. A method for providing a magnetic resonance (MR) scan recommendation, comprising:
receiving, via one or more first interfaces, input data with respect to a medical condition of a patient to undergo an MR scan;
receiving, via one or more second interfaces, evaluation data comprising classification data that classifies anomalies detected in the received input data;
automatically determining, via processing circuitry, a next recommended MR protocol to be executed in accordance with the MR scan of the patient based on the evaluation data; and
transmitting the next recommended MR protocol to an MR scan system to cause the MR scan system to record MR image data from the patient using the next recommended MR protocol.

2. The method according to claim 1, wherein the act of automatically determining the next MR protocol is based on using a machine learning model that includes at least one of a convolutional neural network, a fully connected neural network, a shallow neural network model, a deep neural network model, a decision tree, a random forest, a support vector machine, an expert system, and a classical model algorithm.

3. The method according to claim 1, further comprising:
determining, via processing circuitry, a first possible scan workflow direction as part of the evaluation data by mapping topics based on extracted keywords in the input data onto probability values for a probability of the next recommended MR protocol.

4. The method according to claim 3, wherein the act of automatically determining the next MR protocol comprises:
determining a second possible scan workflow direction by mapping the classification data onto probability values for a probability of the next recommended MR protocol; and
providing the next recommended MR protocol based on the second possible scan workflow direction the first possible scan workflow direction.

5. The method according to claim 1, further comprising:
determining, via processing circuitry, the evaluation data by:
detecting anomalies in the input data; and
determining the classification data based on the detected anomalies.

6. The method according to claim 5, wherein the input data comprise image data, and
wherein the act of detecting the anomalies in the input data comprises detecting the anomalies in the image data.

7. The method according to claim 6, wherein the input data further comprise image-derived metrics based on the image data, and
wherein the act of detecting the anomalies in the input data further comprises detecting the anomalies in the image-derived metrics.

8. The method according to claim 5, wherein the input data further comprise electronic health record information and/or user interface input data, and
wherein the act of determining the evaluation data comprises:
extracting keywords from the electronic health record information and/or the user interface input;
determining topics based on the extracted keywords; and
determining a first possible scan workflow direction by mapping the topics onto probability values for a probability of the next recommended MR protocol.

9. The method according to claim 1, further comprising:
detecting, via processing circuitry, anomalies in the generated MR image data;
determining, via processing circuitry, further classification data based on the detected anomalies in the generated MR image data; and
repeating the act of receiving additional evaluation data including further classification data from subsequent MR scans, automatically determining a next recommended MR protocol, and transmitting the next recommended MP protocol to the MR scan system until an abort criterion is attained.

10. A system for recommending a magnetic resonance (MR) scan of a patient, comprising:
an input interface configured to receive input data with respect to a medical condition of the patient to undergo an MR scan;
scan recommendation processing circuitry configured to:
receive evaluation data comprising classification data that classifies anomalies detected based upon the received input data with respect to a medical condition of a patient; and
automatically determine a next recommended MR protocol to be executed in accordance with the MR scan of the patient based on the evaluation data; and
an output interface configured to transmit the next recommended MR protocol to an MR scan system to cause the MR scan system to record MR image data from the patient using the next recommended MP protocol.

11. The system according to claim 10, wherein the scan recommendation processing circuitry comprises a machine learning model including at least one of: a convolutional neural network (CNN), a fully connected neural network (FCNN), a shallow neural network model, a deep neural network model, a decision tree, a random forest, a support vector machine, an expert system, and a classical algorithm.

12. The system according to claim 10, wherein the scan recommendation processing circuitry is configured to determine a first possible scan workflow direction as part of the evaluation data by mapping topics based on extracted keywords in the input data onto probability values for a probability of the next recommended MR protocol.

13. The system according to claim 12,
wherein the scan recommendation processing circuitry comprises:
mapping circuitry configured to determine a second possible scan workflow direction by mapping the classification data onto probability values for a probability of the next recommended MR protocol; and
final protocol recommendation circuitry configured to provide the next recommended MR protocol based on the second possible scan workflow direction and the first possible scan workflow direction.

14. The system according to claim 10, further comprising:
evaluation circuitry configured to detect the anomalies in the input data, and to determine the classification data based on the detected anomalies.

15. The system according to claim 14, wherein the input data comprise image data, and
wherein the evaluation circuitry is further configured to detect the anomalies in the input data by detecting anomalies in the image data.

16. The system according to claim 15, wherein the input data further comprise image-derived metrics based on the generated MR image data, and
wherein the evaluation circuitry is further configured to detect the anomalies in the input data by further detecting the anomalies in the image-derived metrics.

17. The system according to claim 12, wherein the input data further comprise electronic health record information and/or user interface input data, and further comprising:
evaluation circuitry configured to extract keywords from the electronic health record information and/or the user interface input data, determine topics based on the extracted keywords, and determine the first possible scan workflow direction by mapping the topics onto probability values for a probability of the next recommended MR protocol.

18. The system according to claim 10, further comprising:
anomaly detection circuitry configured to detect anomalies in the generated MR-image data;
classification circuitry configured to determine classification data based on the detected anomalies in the generated MR-image data; and
iteration circuitry configured to repeatedly receive additional evaluation data including further classification data from subsequent MR scans, automatically determine a next recommended MR protocol, and transmit the next recommended MR protocol to the MR scan system until an abort criterion is attained.

19. A non-transitory computer-readable medium having instructions stored thereon that, when executed by one or more processors of a medical imaging system, cause the medical imaging system to:
receive input data with respect to a medical condition of a patient to undergo an MR scan;
receive evaluation data comprising classification data that classifies anomalies detected in the received input data;
automatically determine a next recommended MR protocol to be executed in accordance with the MR scan of the patient based on the evaluation data; and
cause the next recommended MR protocol to be transmitted to an MR scan system to cause the MR scan system to record MR image data from the patient using the next recommended MR protocol.
